(19) United States
(12) Patent Application Publication
(10) Pub. No.: US 2018/0066132 A1
(43) Pub. Date: Mar. 8, 2018

(54) CONDUCTIVE POLYMERIC COMPOSITIONS AND APPLICATIONS

(71) Applicant: The Regents of the University of Colorado, A Body Corporate, Denver, CO (US)

(72) Inventors: Devatha P. Nair, Denver, CO (US); Malik Y. Kahook, Denver, CO (US); Zefram Marks, Boulder, CO (US); Sean Shaheen, Denver, CO (US); Robert R. McLeod, Boulder, CO (US)

(21) Appl. No.: 15/563,764

(22) PCT Filed: Mar. 31, 2016
(86) PCT No.: PCT/US16/25325

§ 371 Date: Oct. 2, 2017

Related U.S. Application Data
(60) Provisional application No. 62/142,728, filed on Apr. 3, 2015.

(51) Int. Cl. C08L 41/00; C08L 39/04; C08L 79/02; C01G 9/02

(52) U.S. Cl. CPC C08L 41/00; C08L 39/04; A61F 2/14; C01G 9/02; C08L 79/02

(57) ABSTRACT

The disclosed technology is directed to conductive polymeric compositions, and methods of manufacturing and using conductive polymer compositions. Specifically, the disclosed technology includes customizing conductive compositions, including conductive polymers and nanogels, with a range of physical and mechanical properties tailored to various applications, including drug delivery, contrast for medical imaging (e.g., optical coherence tomography electrochromics), smart lenses, etc.

### CROSS-REFERENCE

This PCT application claims priority to U.S. provisional application 62/142,728, filed Apr. 3, 2015. For U.S. purposes, this application is a continuation application of U.S. provisional application 62/142,728, filed Apr. 3, 2015.

### TECHNICAL FIELD

This disclosure relates generally to conductive polymeric compositions, particularly, to conductive polymeric compositions that reversible change their absorption.

### STATEMENT REGARDING FEDERALLY SPONSORED RESEARCH OR DEVELOPMENT

None.

### BACKGROUND

Medical devices have a diverse array of surface properties. In order to perform acceptably, and, e.g., to minimize the trauma to the patient, not be rejected, etc., the device and its surface are designed with the application in mind. For example, many medical devices require a combination of properties such as strength, thermal stability, structural stability, flexibility, opacity, radio-opacity, storage stability, lubricity, stability to sterilization treatment, etc., and of course, biocompatibility, in order to be effective for their intended purpose. Some devices need to be not just biocompatible but also either hydrophilic or hydrophobic. For example, some devices for ophthalmic purposes need to be clear and/or have a proper index of refraction.

Material selection is thus very important to the therapeutic efficacy of many medical devices since the properties of the materials used often dictate the properties of the overall device. However, the range of properties available from one, or even a combination of, materials) is often not as broad as would be desired in medical device applications. As a result, many medical devices need to be manufactured from a combination of materials, processed in a specific manner, coated, and/or subjected to other treatments, in order to exhibit the desired and/or required characteristics.

### SUMMARY

The present disclosure is directed to conductive polymeric compositions, and methods of manufacturing and using conductive polymer compositions. Specifically, the disclosed technology includes customizing conductive polymeric compositions with a range of physical and mechanical properties tailored to various applications, applications such as drug delivery, contrast for medical imaging (e.g., optical coherence tomography electrochromics smart lenses, and conductive polymer nerve growth.

The conductive polymeric compositions can have a first absorption at a first time and a second absorption at a second time; in some implementations, the difference in the first time and the second time is an applied voltage. In such an implementation, the composition has a first absorption at a first voltage level and a second (different) absorption at a second voltage level. The (reversible) absorption spectrum shift is due to an electrochemical reaction in the composition facilitated by the applied voltage.

These and various other features and advantages will be apparent from a reading of the following detailed description.

### BRIEF DESCRIPTION OF THE DRAWING

FIG. 1 is a schematic diagram of a microfluidic ion transport test mask illustrating ionic transport through conductive polymer thin film.

FIG. 2 is another schematic diagram of a microfluidic ion transport test mask illustrating ionic transport through conductive polymer thin film.

FIG. 3 is schematic diagram of an organic electrochemical transistor.

FIG. 4 is a schematic diagram of an example lacrimal implant device.

FIG. 5 is a chart of example glaucoma drugs.

FIG. 6 is a schematic diagram of an example environmental electrolyte, electrochromic device.

FIG. 7 is a schematic diagram of another example environmental electrolyte, electrochromic device.

FIG. 8 is a schematic diagram of yet another example environmental electrolyte, electrochromic device.

FIG. 9 is a graphical representation of ion pumping current vs. time.

### DETAILED DESCRIPTION

Iontronics are devices utilizing conductive polymers and other materials that interface between electrical and ionic environments. Electro-ionic interactions occur at time scales of human physiological processes, and have a rich and biocompatible chemistry. Iontronics can interface with the aqueous, ionic environment of the body, and include transistors for processing signals and computing logic operations, optics for modulating and diffracting light, and provide transport of ionic species for drug delivery. Computing can be performed in vivo in response to internal or external stimulus. Devices embedded in the body can communicate with a device outside of the body to leverage data analytics and powerful algorithms for smart biomedical devices.

The present disclosure describes various conductive polymeric compositions, methods of manufacturing the conductive polymeric compositions, and various applications for conductive polymeric compositions with iontronics. Specifically, the disclosed technology includes customization of conductive polymers, and nanogels, with a full range of physical and mechanical properties for use in various applications. The conductive polymers and nanogels described herein can be synthesized from modified commercially available polymers and customized for desired and/or intended use. This customization provides for actuating, modulating, and controlling a system, and doing so externally from the body with iontronics. The conductive polymers and nanogels described herein can be synthesized to specification or intended use. The conductivity may be provided by the polymeric composition itself, by a metal or metallic additive (e.g., silver, gold) in or on the polymeric composition, or by a conductive oxide (e.g., transparent conductive oxide) present in or on the polymeric composition.

The present disclosure is directed to conductive polymeric compositions that are particularly suited for medical applications such as drug delivery devices, surgical tools, lenses (e.g., intraocular lenses and contact lenses), and other uses. It is noted that the phrases “conductive polymeric compositions,” “conductive compositions,” and variations thereof, encompass conductive nanogels, unless specifically indicated otherwise.

The conductive compositions are particularly suited as coatings on, fillers in, or fillers in coatings on ophthalmic devices (e.g., lenses), since in some implementations the conductive compositions are transparent. The conductive compositions are also particularly suited as a therapeutic agent delivery conduit. The conductive compositions enable a specific parameter of a device or material to be altered (such as the refractive index of a lens, equilibrium water content, tackiness, flexibility, durability, etc.) without impacting any other material properties. For example, the conductive compositions can be configured for storing and/or delivering therapeutic and/or active molecules or agents, such as biological and/or non-biological active molecules (e.g., drugs, biologics (e.g., peptides, apatamers etc.)), with or without an associated coating that controls rate of delivery of the therapeutic or active molecule to the surrounding tissue. The conductive compositions can also be specifically designed with a charge, such as anionic or cationic.

The conductive polymers described herein can include an electrically conductive polymer or combination of polymers selected from polyfluorenes, polyphenylenes, polypyrenes, polyazulenes, polynaphthalenes, polypyrroles, polycarbazoles, polyindoles, polyazepines, polyanilines, polythiophenes, polyacetylenes, and copolymers thereof. In a more particular implementation, the electrically conductive polymer is selected from poly(3,4-ethylenedioxythiophene) (PEDOT), polyaniline (PANT), polypyrrole (PPy), poly(phenylene vinylene) (PPV), poly(arylene), polyspirobifluorene, poly(3-hexylthiophene) (P3HT), poly(o-methoxyaniline) (POMA), poly(o-phenylenediamine) (PPD), or poly(p-phenylene sulfide). The conductive polymers can also include functionalized PEDOT with additional functional groups on the end of the polymer chain (e.g., PEDOT-TMA (PTMA), PEDOT-PEG, and PEDOT block PEG).

In several examples in this disclosure, the conductive polymer poly(3,4-ethylenedioxythiophene):polystyrene sulfonate (PEDOT:PSS) is used. PEDOT is an organic semiconductor while PSS is a “dopant” material (provides the charge carrier to the semiconductor, making the layer electrically conductive) as well as providing ancillary benefits such as increased solubility. PEDOT:PSS is solution processable (e.g., for spin coating thin films), has good electrical conductivity (10-1000 S/cm), is hydroscopic, ionically conductive, electrochemically active, and electrochromic. However, other polymer compositions customized with improved and optimized properties may be used in the disclosed technology.

For example, a polymer composition may include a PEDOT derivative, a nanogel, or a combination of both, that could optimize one material property at the expense of another. For example, an implementation could have reduced electrical conductivity with increased ionic conductivity, and the polymer composition can be used as either an electrolyte or as an ion transport material.

In some implementations, customization of a system can include incorporation of a customized nanogel. Nanogels are polymer ensembles that can be used to mix hydrophobic and hydrophilic materials, with novel chemical, mechanical, and optical properties. Nanogels can be charged, behave like ions in solution, and be transported inside conductive polymer films. Nanogels can be cross-linked to form solid layers with large water content, opening up the possibility of novel solid electrolytes and other device layers. By using nanogels, in some implementations, either with conductive polymers or in place of conductive polymers, there may be more system capabilities (e.g., nanogels can be used as an outside bacterial coating, texturing a surface during integration).

In some implementations the reactive pendent group of the nanogel is an acrylate or an isocyanate. In some implementations, the nanogels are siloxane nanogels, having been formed from a composition including a siloxane acrylate. In some implementations, the composition includes a siloxane acrylate and an acrylate, such as a urethane-based acrylate. Urethane-based acrylate(s) enable secondary interactions (reversible hydrogen bonding) within the composition. In some implementations, other monomer, polymer or copolymer material(s) may be used in place of the urethane-based acrylate(s), as long as they provide secondary interactions (reversible hydrogen bonding) in the composition and meet the criteria for an intraocular lens. In other implementations, the composition includes a siloxane acrylate and a hydrophilic monomer, such as a hydrophilic acrylate.

In one implementation of the disclosed technology, a system comprises an electrical conductor, at least two ionic conductors, and an electrochromic material, all of which can be customized for predetermined specifications and intended uses. The electrical conductor conducts electricity for electrodes, wires, etc. The electrical conductor can include synthesized or modified conductive polymers (e.g., PEDOT:PSS, P3HT, polypyrrole, polyaniline, polythiophene), metals, and transparent conductive oxides (e.g., ITO, ZnO).

For example, some polymers can electrochemically transport ions and interact with a charged ionic drugs to store or transport the drugs through polymer channels. The disclosed technology provides customization of conductive polymers to the ionic charge on that specific drug. In another example, the refractive index, absorption, or film thickness of a polymer or nanogel film can be changed by the electrochemical interaction with an electrolyte. Modulating these effects can result in dynamic changes to the optical, mechanical, and electrical properties of the film (e.g., electrochromic effects).

The ionic conductors provide an environment for ions used between the electrochemical electrodes, and can be a structural material in some implementations. The ionic conductors can be an aqueous solution (e.g., salt water,) a gel electrolyte, and/or a solid (e.g., cross-linked polymer, nanogel, etc.) The system can include another ionic conductor, an ion transport layer or media to transport ions or large charged molecules (e.g., therapeutic drugs). The system also includes an electrochromic material to facilitate changes of optical properties using a electrochemical reaction and changes in absorption spectrum (e.g., color), index of refraction, physical swelling of film, polarization, etc.

In one implementation, an application for the conductive polymers in the disclosed technology includes integrating a conductive polymeric composition with a medical device, such as a coating, for drug delivery. The delivery may be passive delivery, e.g., via diffusion from the coating, or may be active, e.g., via arbitrary pharmacokinetics. Electro-ionically switched biomedical devices can directly control charged chemical species, including drugs. Ionically charged molecules can be stored and transported inside polymer thin films. The transport and controlled release of charged drugs can be included in these ionic devices to create an integrated therapeutic system of sensing, computing, communication and drug delivery.

In another implementation, a conductive polymeric coating can be applied to a medical device used for iontophoresis in which a small electric charge is used to deliver one or more active agents to one or more tissues of a patient, for example, for transdermal delivery of one or more active agents. In another implementation, a conductive polymeric coating can be used in connection with implantable electronic active agent delivery systems to administer active agents (e.g., to fight bacterial infection).

In the following description, reference is made to the accompanying drawing that forms a part hereof and in which are shown by way of illustration at least one specific implementation. The following description provides additional specific implementations. It is to be understood that other implementations are contemplated and may be made without departing from the scope or spirit of the present invention. The following detailed description, therefore, is not to be taken in a limiting sense. While the present invention is not so limited, an appreciation of various aspects of the invention will be gained through a discussion of the examples provided below.

As used herein, the singular forms “a”, “an”, and “the” encompass implementations having plural referents, unless the content clearly dictates otherwise. As used in this specification and the appended claims, the term “or” is generally employed in its sense including “and/or” unless the content clearly dictates otherwise.

Spatially related terms, including but not limited to, “lower”, “upper”, “beneath”, “below”, “above”, “on top”, etc., if used herein, are utilized for ease of description to describe spatial relationships of an element(s) to another. Such spatially related terms encompass different orientations of the device in addition to the particular orientations depicted in the figures and described herein. For example, if a structure depicted in the figures is turned over or flipped over, portions previously described as below or beneath other elements would then be above or over those other elements.

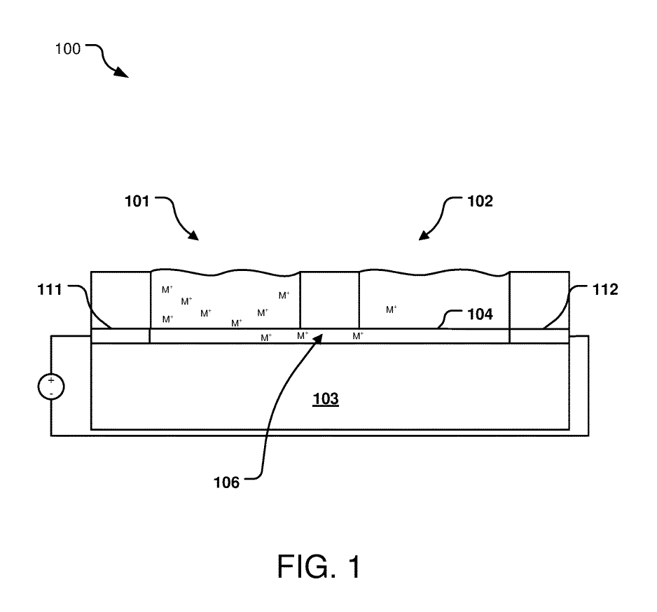

FIG. 1 is a schematic diagram of ionic transport through a conductive polymer thin film in a microfluidic ion transport test mask. A test mask 100 has two wells of electrolyte(s), well 101 and well 102, which are shown over an ion transport layer 104. Well 101 has a high concentration of ions, depicted as seven “M+” ions. Well 102 has a low concentration of ions, depicted as one “M+” ion. By applying an electric field across electrodes 111, 112, the ions from a well (e.g., well 101) can be pulled through a channel 106, through the ion transport layer 104, and into another well (e.g., well 102). A reservoir of active, e.g., drugs, can be transported through membranes and controlled electrically for delivery to the body with precise dosage control and timing. The electrolyte(s), the electrodes 111, 112, and any other polymeric features of the test mask 100 can be customized based on the active (and its charge) that needs to be delivered.

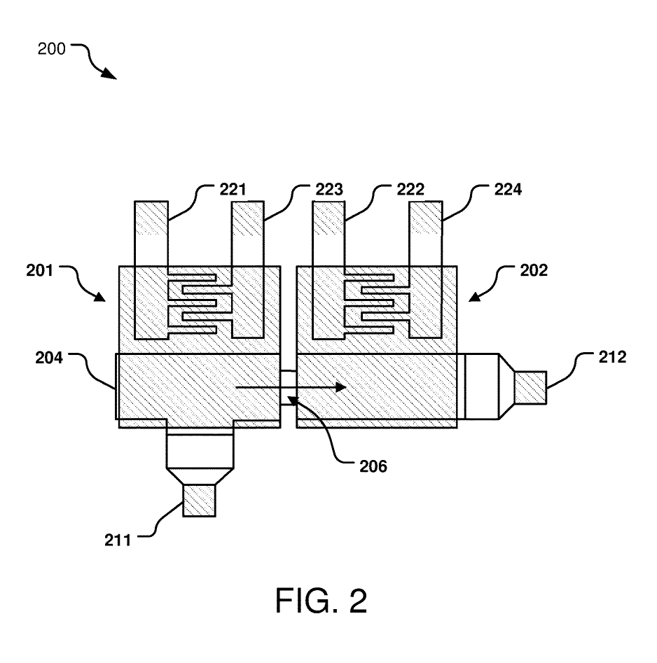

FIG. 2 shows a second schematic diagram of ionic transport through conductive polymer thin film in a microfluidic ion transport test mask 200. Similar to the test mask 100 of FIG. 1, a test mask 200 has two wells of electrolyte(s), well 201 and well 202, connected via an ion transport layer 204. The ion transport layer 204 also connects a first electrode 211 with a second electrode 212. The test mask 200 also includes two conductivity sensors in each well, i.e., sensors 221, 223 in the first well 201 and sensors 222, 224 in the second well 202. By applying an electric field across electrodes 211, 212, the ions from a well (e.g., well 201) can be pulled through a channel 206, through the ion transport layer 204, and into another well (e.g., well 202).

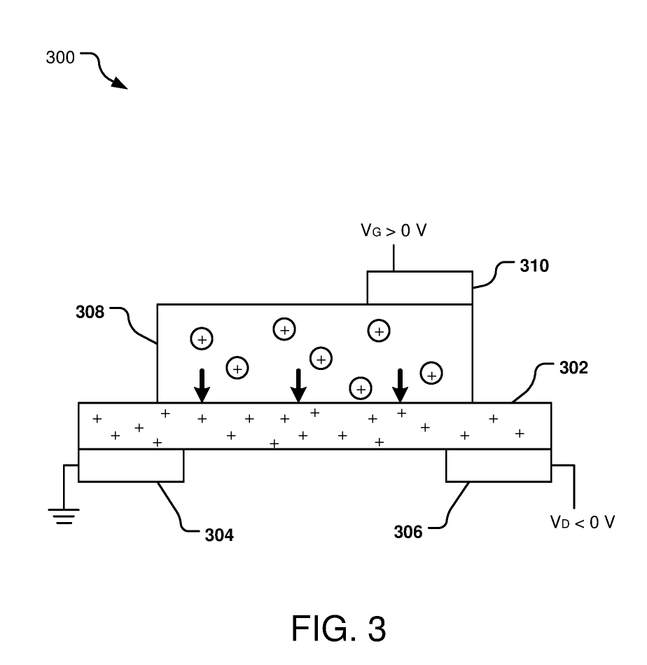

FIG. 3 shows an organic electrochemical transistor 300 that can be made with a conductive polymeric composition. The transistor 300 has an electrical channel 302 formed by a conductive polymeric composition, such as PEDOT:PSS. The channel 302 connects a source electrode 304 (e.g., gold) with a drain electrode 306. Present on the channel 302 is an electrolyte gel 308 that electrically connects the channel 302 with a gate 310. Current flow through the channel 302 from the source 304 to the drain 306. Ions within the electrolyte 308 migrate toward the channel 302 upon application of a voltage, thus depositing ions on the channel 302 and altering the properties of the channel 302.

In another implementation, a conductive polymer composition can be used in or on devices implanted in a user. In some implementations, the device is a medical device configured to transfer electrical energy (e.g., radiofrequency energy (RF)) to another device implanted in the patient. In other implementations, the device is a medical device that is implanted in the patient and operably connected to a device that provides electrical energy; examples of such devices include stimulators (e.g., neurostimulators, cardiac stimulators), and other devices that utilize electrical leads or contacts to provide electrical stimulus to the patient. In other implementations, the device is an implanted ophthalmic device, such as an intraocular lens.

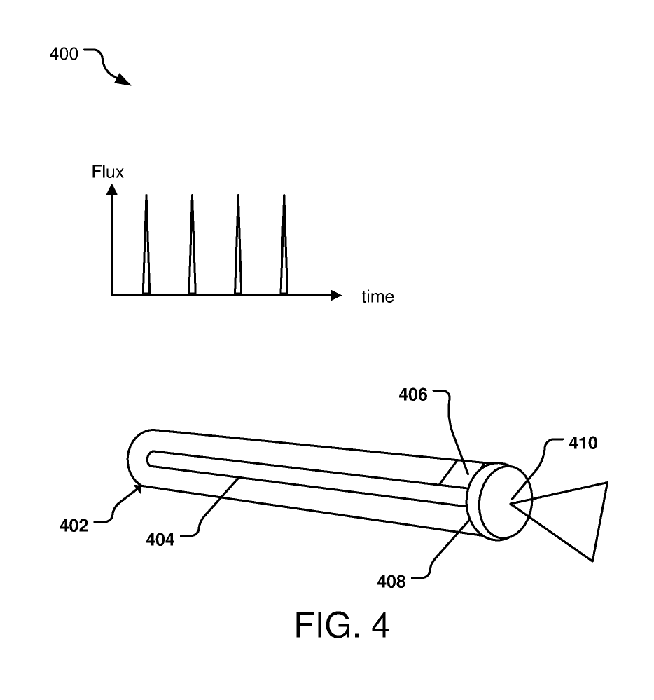

FIG. 4 is a schematic diagram of a lacrimal implant 400 for drug delivery, and the inset diagram in FIG. 4 illustrates drug molecule flux versus time for nominal operation.

This implementation of the implant 400 has body 402 having a microfluidic channel and drug reservoir 404, OECT control circuits 406, a control electrode 408, and an ionically conductive membrane 410, all of which can be customized to an ionically charged drug. Example dimensions for the implant 400 are about 1 cm in length and about 1 mm in diameter. The microfluidic channel and drug reservoir 404 can hold a charged drug (e.g., an ophthalmic drug). The ionically conductive membrane 410 (e.g., comprising PEDOT or other customized composition) can be used to control the release of a drug with applied voltage on the control electrode 408. The control circuits 406 (e.g., inorganic or organic semiconductors) provide power, control, and/or communication in the system.

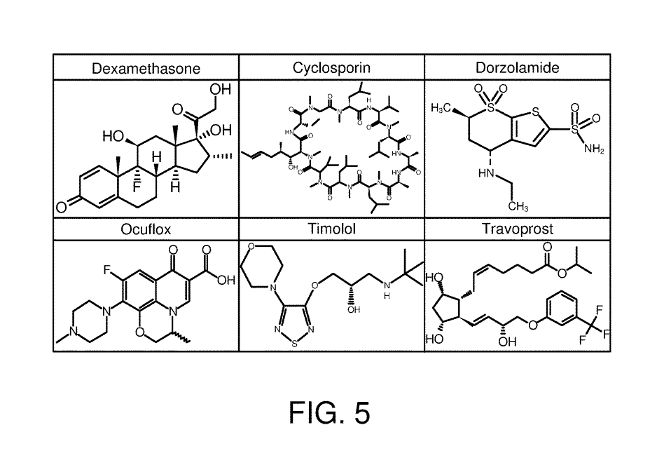

FIG. 5 is a table of examples of glaucoma drugs that could be used in an ionic drug delivery device, such as lacrimal implant 400, an intraocular lens, or even a contact lens. These and other drugs can be incorporated into or onto conductive polymer compositions that form or are part of an ophthalmic device.

In another implementation, an application with conductive polymer compositions involves a device (e.g., surgical tool) with at least one electrically conductive layer on the surface. For example, in optical coherence tomography (OCT), which is a bio-imaging technique used for imaging the eye during optical surgery to obtain a profile view and a top-down microscope view, metal tools block the OCT and conventional polymeric surgical tools are difficult to see with OCT because of their low absorption and low contrast in the near-IR. The present disclosure includes applying electrochromic conductive polymer coatings on surgical tools to shift the absorption spectrum or to facilitate absorption and thus visualization of these tools in OCT. By applying an electric field or a voltage, the absorption properties can be changed from transparent to opaque on the OCT image. Additionally, the coating water content, thickness and/or refractive index can be modulated by the ion concentration. The practitioner, e.g., surgeon, can turn off the OCT to see the ion environment. In another implementation, an electric field can be applied to the OCT, so that the coated tool glows brightly and the surgical tool becomes visible or invisible, as desired.

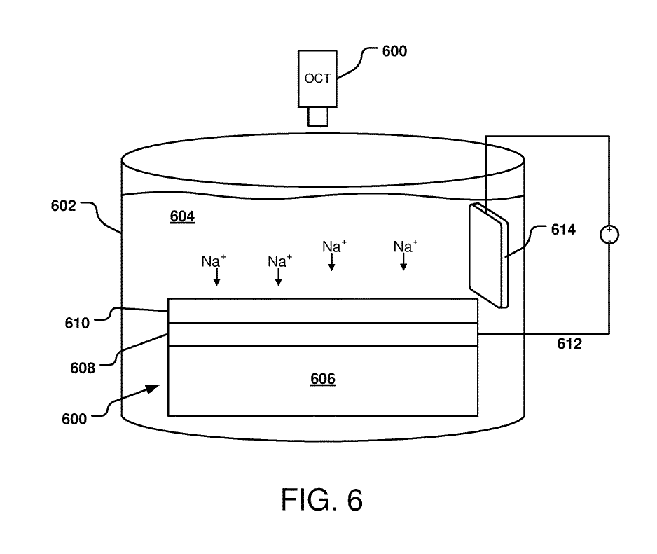

Example polymer coating applications on an OCT tool is illustrated in FIGS. 6 through 8, which are schematic diagrams of example environmental electrolyte, electrochromic devices. In these implementations, a PEDOT composition, which has an electrochromic effect, is coated onto the surface of an OCT tool. The three figures illustrate possible variations to a system. For example, there may be different layers implemented, such as an electrolyte layer and a PEDOT layer in the device. Some the layers can be optimized for electrochemical spectroscopy, and other layers can be optimized for conductivity in electrodes.

In FIG. 6, a vessel 602 retains an electrolyte 604 in which is placed an OCT tool 600. The OCT tool 600 has a base substrate 606 and is electrically connected to a working electrode 612 via a transparent conductive oxide 608 (e.g., ITO, ZnO) present on the substrate 606 and to a counter electrode 614 that is submerged in the electrolyte 604. Also present on the substrate 606, illustrated as present over the conductive oxide 608, is a conductive polymeric composition layer 610, for example, a PEDOT:PSS layer.

As ions (shown as, e.g., Na+ ions) from the electrolyte 604 are pumped and deposited onto the PEDOT:PSS layer 610, the absorption changes and as a result, there is an absorption shift from the infrared to the invisible, causing the layer 610 to physically darken. As ions are pumped, the absorption shift changes their optical properties in the OCT tool 600.

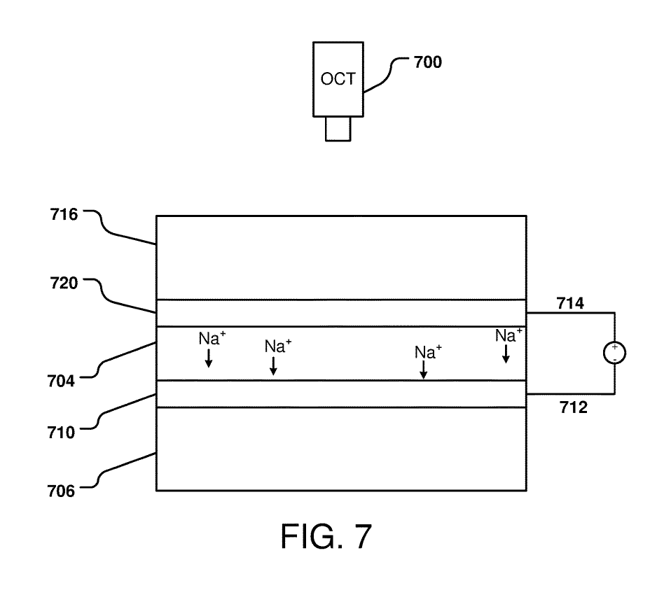

In FIG. 7, an OCT tool 700 has a base substrate 706 and an electroconductive layer 710 formed from a conductive polymeric composition (e.g., PEDOT:PSS) and a second substrate 716 and second conductive polymeric composition layer 720 (e.g., PEDOT:PSS). Between the conductive polymeric composition 710 and the second a conductive polymeric composition 720 is an electrolyte 704. The conductive polymeric composition 710 is electrically connected to a working electrode 712 and the second conductive polymeric composition layer 720 is electrically connected to a counter electrode 714.

Ions (shown as, e.g., Na+ ions) from the electrolyte 704 are deposited onto the conductive polymeric composition layer 710, so that the absorption changes and an absorption shift from the infrared to the invisible occurs. As ions are deposited, the absorption shift changes the optical properties in the OCT tool 700.

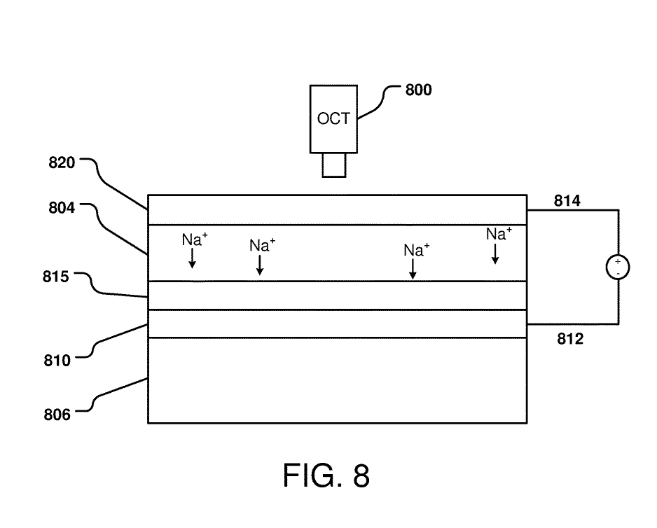

In FIG. 8, an OCT tool 800 has a base substrate 806 with a first electroconductive layer 810 formed from a conductive polymeric composition (e.g., PEDOT:PSS) or a transparent conductive oxide (e.g., ITO, ZnO) and a second electroconductive layer 815. Also present is a second conductive polymeric composition layer 820 (e.g., PEDOT:PSS). Between the first electroconductive layer 810 and the second electroconductive layer 820 is an electrolyte 804. The first electroconductive layer 810 is electrically connected to a working electrode 812 and the second first electroconductive layer 820 is electrically connected to a counter electrode 814.

Ions (shown as, e.g., Na+ ions) from the electrolyte 804 are deposited onto the second electroconductive layer 815, which changes the optical properties in the OCT tool 800.

In some implementations, the optical properties of the conductive compositions can be dynamically changed using electro-ionic signals. The optical properties can be controlled by electrochemically modulating the water content, refractive index, the absorption spectrum, or the thickness of the polymer layer or the nanogel layer. These conductive compositions can be used to make refractive or diffractive optical elements including diffraction gratings, Bragg holograms, diffractive optical elements, waveplates or other retarders, waveguides, prisms or lenses with dynamic properties. One example is a lens whose focal power can be modified for glasses or contact lenses. Combining these optical actuators with the computing, sensing, and communication interface capabilities of ionic transistors further enhances their utility.

Another implementation for an application for conductive polymer compositions is for nerve growth/regeneration. Electrical stimulation has been shown to be useful for regeneration of nerves where it promotes the growth of axons. Electroconductive coatings can be useful to enhance the performance of devices designed to stimulate nerve growth by improving the tissue interface. Electroconductive coatings can also be useful as part of scaffolds intended to guide nerve regeneration.

Organic electrochemical transistors (OECTs) are electrochemically actuated and have high trans-conductance. As such, they are a built-in amplifier for a biological signal. An interface can be functionalized to obtain a variety of responses from the ionic signaling of neurons. The high transconductance of OECTs means that small biological signals can be amplified in vivo, increasing the sensitivity of sensors. Neurons can be grown on surfaces patterned with customized conductive polymer electrodes and used as a bioelectrical interface. In one implementation, OECTs can be synthesized to connect to the neurons and directly detect their ionic signaling.

In one implementation, the disclosed technology includes conductive photo-resist resins. By mixing a highly conductive PEDOT:PSS polymer with functionalized PEDOT:TMA or PEDOT nanogels, films can be made to cross-link to form a strong, robust polymer layer while still being conductive. The crossed-linked PEDOT:TMA or other functionalized polymer (e.g., PEG additive to PEDOT:PSS) forms a polymer matrix that holds together the high conductivity PEDOT:PSS polymer chains. By adding a photoinitiator (e.g., TPO-L) the solution can be polymerized with UV light. By coating (e.g., spin-casting) a thin film layer of this solution, photolithography techniques can be used to selectively expose regions of the film to crosslink. As a result, the film behaves as a “negative tone” photoresist. The unexposed and uncrosslinked parts of the film can be removed with post-exposure treatment (e.g., solvents, plasma, etc.). With this technology, various patterns of nanogels and other polymers can be formed.

In some implementations of coating the conductive compositions onto devices, such as surgical tools or OCT tools or implantable medical devices, there is no covalent adherence and the coatings may not be stable. The conductive compositions can be designed to covalently adhere to the devices, such as to substrates or other layers and coatings on the devices. In addition, the substrate or surface of the device can be treated with a functionalized coating (e.g., silane-based chemistry) to form functional groups that can be cross-linked with the conductive composition (e.g., PEDOT:TMA) to improve adhesion.

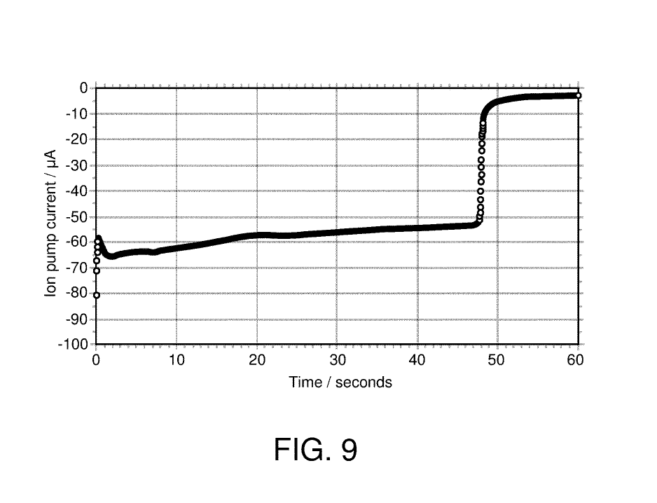

FIG. 9 is a graph of the electrode current vs. time during an ion transport run through a conductive composition; electric current was measured as function of time in response to an applied voltage. Resistivity was measured before and after the run as a proxy for ion concentration. The conductivity increased (and thus the resistivity decreased) due to the voltage-driven ion concentration, thereby increasing the electric current measured and changing the material's ability to conduct an electric current. The measurement revealed an increase in conductivity, and thus an increase in ionic concentration.

The conductive polymers may be polymerized by any conventional method using ingredients (e.g., monomers) that result of which is conductivity. Examples of electrically conductive compositions include, but are not limited to PEDOT, PEDOT:PSS, PEDOT:TMA, compositions formed from DMAEMA and PTMA or TMA.

In indicated above, the conductive polymer compositions may be nanogels. Both the conductive polymers and nanogel compositions have a polymeric backbone with at least one reactive group. The backbone is, or is formed from a polymer that is, hydrophilic, hydrophobic, amphiphilic, or conductive. In most implementations, the reactive group is an acrylate, but could alternately be an isocyanate or alcohol.

In some implementations, the polymeric composition includes at least one siloxane acrylate. The polymeric composition may additionally include at least one acrylic copolymer, such as a urethane-based acrylic copolymer. The siloxane acrylate may be a silicon-containing acrylate, diacrylate, triacrylate, tetracrylate, etc. The acrylate may be, for example, aliphatic or aromatic. Examples of suitable siloxane acrylates include silicone diacrylate (e.g., commercially available under the trade name “Ebecryl 350”), [tris(trimethylsiloxy)silyl]propyl methacrylate, and 3-(trimethoxysilyl)propyl methacrylate.

The polymeric composition that will form the conductive composition can include other copolymer or monomer materials, including thermosetting materials (e.g., phenolics, epoxies, urea-formaldehydes, melamine formaldehydes, and the like) or thermoplastic materials (e.g., polyamides (nylon), polyethylene, polypropylene, polyesters, polyurethanes, polyetherimide, polysulfone, polystyrene, acrylonitrile-butadiene-styrene block copolymer, acetal polymers, polyvinyl chloride, and the like). The composition can include other materials such as, for example, photoinitiator(s), plasticizer(s), catalyst(s), accelerator(s), activator(s), coupling agent(s), lubricant(s), wetting agent(s), surfactant(s), UV blocker(s), UV stabilizer(s), and/or complexing agent(s). The polymeric composition can be 100% solids; that is, no solvent is present. In some implementations, however, the polymeric composition is composed of only acrylate(s).

The compositions can be electrically conductive; additionally, the compositions can be charged, either anionic or cationic.

The above specification, examples, and data provide a complete description of the structure, features and use of exemplary implementations of the invention. Since many implementations of the invention can be made without departing from the spirit and scope of the invention, the invention resides in the claims hereinafter appended. Furthermore, structural features of the different implementations may be combined in yet another implementation without departing from the recited claims.

1. An electrically conductive polymeric composition having a polymeric backbone and a reactive pendant group, the polymeric backbone being hydrophilic, hydrophobic, amphiphilic, or electrically conductive, the conductive polymeric composition having a first absorption state at a first voltage level and a second absorption state at a second voltage level.

2. The polymeric composition of claim 1, further comprising at least one of a conductive polymer, a metal, and a transparent conductive oxide.

3-21. (canceled)

22. The polymeric composition of claim 2, wherein the transparent conductive oxide is ITO or ZnO.

23. The polymeric composition of claim 1, wherein the reactive pendant group is an acrylate, methacrylate, isocyanate, or alcohol.

24. The polymeric composition of claim 1, wherein the polymeric backbone is electrically conductive.

25. The polymeric composition of claim 24, wherein the polymeric backbone comprises at least one of PEDOT:PSS, P3HT, polypyrrole, and polyaniline.

26. The polymeric composition of claim 1 being a carrier for a biological molecule.

27. The polymeric composition of claim 1 as a contrast agent.

28. The polymeric composition of claim 1 on a surface of a device used for transferring electrical energy.

29. The polymeric composition of claim 28, wherein the device is a sensor for transferring electrical energy.

30. The polymeric composition of claim 1 on a surface of a device used to sense electrical activity of a body of a user.

31. The polymeric composition of claim 1 present as a thin film coating.

32. A biological sensor having a surface comprising an electrically conductive polymeric composition comprising a polymeric backbone and a reactive pendant group, the polymeric backbone being hydrophilic, hydrophobic, amphiphilic, or electrically conductive, the conductive polymeric composition having a first absorption state at a first voltage level and a second absorption state at a second voltage level.

33. The polymeric composition of claim 32, wherein the reactive pendant group is an acrylate, methacrylate, isocyanate, or alcohol.

34. The polymeric composition of claim 32, wherein the polymeric backbone is electrically conductive.

35. The polymeric composition of claim 32, wherein the polymeric backbone comprises at least one of PEDOT:PSS, P3HT, polypyrrole, and polyaniline.

36. The biological sensor of claim 32, wherein the electrically conductive polymeric composition is present as a film on the sensor.

37. The biological sensor of claim 32, wherein the electrically conductive polymeric composition is a carrier for a biological molecule.

38. The biological sensor of claim 32, wherein the sensor senses electrical activity of a body of a user.
